(19) United States
(12) Patent Application Publication
(10) Pub. No.: US 2026/0028609 A1
(43) Pub. Date: Jan. 29, 2026

(54) PROTEASE VARIANTS WITH IMPROVED STORAGE STABILITY

(71) Applicant: Henkel AG & Co. KGaA, Dusseldorf (DE)

(72) Inventors: Fabian Falkenberg, Neuss (DE); Christian Degering, Erkrath (DE); Pascal Mackenberg, Dusseldorf (DE); Margret van Lier, Hilden (DE)

(21) Appl. No.: 19/265,451

(22) Filed: Jul. 10, 2025

(30) Foreign Application Priority Data
Jul. 26, 2024 (DE) 102024207057.2

(51) Int. Cl. C12N 9/54; C11D 3/386

(52) U.S. Cl. CPC C12N 9/54; C11D 3/386; C12Y 304/21062

(57) ABSTRACT

The invention relates to proteases that exhibit proteolytic activity and comprise an amino acid sequence that is at least 70% identical to the amino acid sequence indicated in SEQ ID NO:1 over its entire length, wherein the proteases, in each case based on the numbering in accordance with SEQ ID NO:1, have at least one amino acid substitution selected from the group consisting of N121F, A194C, A209V, T218I, N237P and N237W in at least one of the positions corresponding to positions 121, 194, 209, 218 and 237. Such proteases are suitable for use in washing and cleaning agents, in particular textile washing agents, and have improved storage stability in comparison with a reference protease. The invention also relates to the use of said proteases and methods in which they are used, and to washing and cleaning agents containing said proteases, in particular textile washing agents.

### CROSS-REFERENCE TO RELATED APPLICATIONS

The instant application claims the benefit of German Patent Publication No. 102024207057.2, filed on Jul. 26, 2024, which is incorporated by reference in its entirety herein.

### SEQUENCE LISTING

The Instant Application contains a Sequence Listing which has been submitted electronically in XML format and is hereby incorporated by reference in its entirety. Said XML copy, created on Jun. 4, 2025 is named “HN10054US” and is 2,245 bytes in size.

### FIELD OF THE INVENTION

The invention is in the field of enzyme technology. The invention relates to proteases, the amino acid sequence of which has been altered in particular with regard to use in washing and cleaning agents, in particular with regard to textile washing agents, in order to improve their storage stability, and to the nucleic acids coding for them, and to the production thereof. The invention also relates to the use of said proteases and methods in which they are used, and to washing and cleaning agents containing said proteases, in particular textile washing agents.

Proteases are among the technically most important enzymes. For washing and cleaning agents, they are the longest established enzymes and are contained in virtually all modern, high-performance washing and cleaning agents. They cause the degradation of protein-containing stains on the articles to be cleaned. In turn, proteases of the subtilisin type (subtilases, subtilopeptidases, EC 3.4.21.62) are particularly important, which proteases are serine proteases due to the catalytically active amino acids. They act as unspecific endopeptidases and hydrolyze any acid amide bonds within peptides or proteins. Their optimum pH is usually in the distinctly alkaline range. The article “Subtilases: Subtilisin-like Proteases” by R. Siezen, pages 75-95 in “Subtilisin enzymes,” published by R. Bott and C. Betzel, New York, 1996, for example, gives an overview of this family. Subtilases are naturally formed by microorganisms. In particular, the subtilisins formed and secreted by the Bacillus species are the most significant group of subtilases.

Examples of subtilisin-type proteases that are preferably used in washing and cleaning agents are the subtilisins BPN′ and Carlsberg, protease PB92, subtilisins 147 and 309, the alkaline protease from Bacillus lentus, in particular Bacillus lentus DSM 5483, subtilisin DY and the enzymes thermitase, proteinase K and proteases TW3 and TW7, which are to be classified as subtilases, but no longer as subtilisins in the narrower sense, as well as variants of said proteases that have an amino acid sequence which is altered compared to the starting protease. Proteases are altered in a targeted or random manner by methods known from the prior art and are thus optimized for use in washing and cleaning agents, for example. This includes point, deletion or insertion mutagenesis, or fusion with other proteins or protein parts. Thus, appropriately optimized variants are known for most proteases known from the prior art.

In general, only selected proteases are suitable for use in liquid, surfactant-containing preparations in any case. Many proteases do not exhibit sufficient catalytic performance in such preparations or they are not sufficiently stable. For the use of proteases in washing and cleaning agents, therefore, a high catalytic activity and stability under conditions such as are present during a washing process is particularly desirable. Consequently, protease- and surfactant-containing liquid formulations from the prior art are disadvantageous in that the proteases present, under standard washing conditions, do not exhibit satisfactory proteolytic activity and/or are not sufficiently storage-stable, and the formulations therefore do not exhibit optimal cleaning performance on protease-sensitive stains. Protease-sensitive stains are preferably selected from the group consisting of blood, egg (yolk), milk and other protein-containing stains.

There is still a need to improve the storage stability of enzyme-containing, in particular protease-containing, washing and cleaning agents, in particular textile washing agents, in particular with regard to the cleaning performance on protease-sensitive stains, in particular in a temperature range of approximately 20° C. to approximately 40° C. Furthermore, there is still a need to improve the storage stability of the protease in a protease-containing washing and cleaning agent, in particular textile washing agent.

Surprisingly, it has now been discovered that a protease from Halalkalibacter okhensis Kh10-101 or a protease sufficiently similar thereto (in terms of sequence identity) that, based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237, is improved in terms of its storage stability in comparison with a reference protease (wild type in accordance with SEQ ID NO:1), and is therefore particularly suitable for use in washing and cleaning agents, in particular textile washing agents.

The subject matter of the invention is therefore a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237.

A preferred subject of the invention is a protease that exhibits proteolytic activity and has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, in each case based on the numbering in accordance with SEQ ID NO:1.

A further subject of the invention is a method for producing a protease, comprising introducing at least one amino acid substitution in at least one of the positions corresponding to positions 121, 194, 209, 218 and 237, based on the numbering in accordance with SEQ ID NO:1, selected from the group consisting of N121F, A194C, A209V, T218I, N237P and N237W, into a starting molecule that has an amino acid sequence that has at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% or 100% sequence identity with the amino acid sequence indicated in SEQ ID NO:1 over its entire length.

A protease within the meaning of the present patent application therefore comprises both the protease as such and a protease produced using a method according to the invention. All statements relating to the protease therefore relate both to the protease as such, as well as to the proteases produced by means of corresponding methods, and to the corresponding methods, in particular production methods of the protease.

Further aspects of the invention relate to the nucleic acids coding for these proteases, non-human host cells containing proteases or nucleic acids according to the invention, as well as washing and cleaning agents comprising proteases according to the invention, in particular textile washing agents, washing and cleaning methods, and the use of a protease according to the invention in a washing or cleaning agent, in particular textile washing agent, for the removal of at least one protease-sensitive stain.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

Unless indicated otherwise, all percentages are indicated in terms of weight percent (wt. %).

Numerical ranges that are indicated in the format “from x to y” also include the stated values. If several preferred numerical ranges are specified in this format, it is readily understood that any ranges resulting from the combination of the various endpoints are also included.

“At least one,” as used herein, means one or more, i.e., 1, 2, 3, 4, 5, 6, 7, 8, 9, 10, 11, 12, 13, 14 or more.

The term “washing and cleaning agent” or “washing or cleaning agent,” as used herein, is synonymous with the term “agent” and denotes a composition for cleaning textiles and/or hard surfaces, in particular dishes, as explained in the description.

“Approximately,” “about,” or “roughly,” as used herein in reference to a numerical value, refers to the corresponding numerical value±10%, preferably ±5%.

“Substantially free from” means that the composition or the agent contains less than 2 wt. %, preferably less than 1 wt. %, more preferably less than 0.5 wt. %, and particularly preferably less than 0.1 wt. %, of the corresponding substance, based on the total weight of the composition/agent.

“Liquid,” as used herein, includes liquids and gels as well as pasty compositions. It is preferred that the liquid compositions be flowable and pourable at room temperature, but it is also possible for them to have a limit of liquidity.

A substance, e.g., a composition or an agent, is solid in accordance with the definition of the invention if it is in a solid state of aggregation at 25° C. and 1013 mbar.

A substance, e.g., a composition or an agent, is liquid in accordance with the definition of the invention if it is in a liquid state of aggregation at 25° C. and 1013 mbar. Liquid also includes gel form.

“Variant,” as used herein, refers to naturally or artificially generated variations of a native protease which has an amino acid sequence which is modified from the reference form.

An “improvement in the stability of an enzyme” within the meaning of the invention exists if the presence of an amino acid substitution as defined herein causes a protease with such an amino acid alteration to have, after storage in washing and/or cleaning agents, in particular textile washing agents, a higher enzymatic activity of the protease and/or of any further enzymes present in the washing and/or cleaning agent, in comparison with a control preparation comprising a protease without an amino acid alteration according to the invention. After storage in washing and/or cleaning agents, in particular textile washing agents, a protease according to the invention has a higher residual activity than a reference protease (e.g., the wild-type protease), the proteases being treated in the same way, in particular with regard to the storage conditions and the determination of the enzyme activity. Increasingly preferably, storage takes place for at least 1 week, 2 weeks, 3 weeks, or 4 weeks. More preferably, storage takes place at a temperature of 20° C., 25° C., 30° C., or 40° C.

The term “textile” as used herein means any textile material, including yarns, yarn precursors, fibers, nonwovens, natural materials, synthetic materials and all other textile materials, fabrics made from these materials, and products made from fabrics (e.g., garments and other articles). The textile or fabric can be in the form of knits, wovens, denims, nonwovens, felts, yarns and terry cloth. The textile can be based on cellulose, for example natural cellulose fibers such as cotton, flax/linen, jute, ramie, sisal or coconut fibers, or synthetically produced cellulose fibers (e.g., from pulp) such as viscose/rayon, cellulose acetate fibers (tricell), lyocell or mixtures thereof. The textile or fabric can also consist of non-cellulosic fibers, e.g., natural polyamides such as wool, camel, cashmere, mohair, rabbit hair and silk, or synthetic polymers such as nylon, aramid, polyester, acrylic, polypropylene and spandex/elastane or mixtures thereof, as well as mixtures of cellulosic fibers and non-cellulosic fibers. Examples of blends are blends of cotton and/or rayon/viscose having one or more accompanying materials such as wool, synthetic fibers (e.g., polyamide fibers, acrylic fibers, polyester fibers, polyvinyl chloride fibers, polyurethane fibers, polyurea fibers, aramid fibers) and/or cellulose-containing fibers (e.g., rayon/viscose, ramie, flax/linen, jute, cellulose acetate fibers, Lyocell). The fabric can be conventional washable laundry, e.g., soiled household laundry. When the term “fabric” or “garment” is used, it is intended to include the broader term “textiles.”

The present invention is based on the surprising finding by the inventors that amino acid substitutions at the positions described herein bring about improved storage stability of this altered protease in washing and cleaning agents, in particular textile washing agents.

In preferred embodiments, the alterations according to the invention in at least one of the positions corresponding to positions 121, 194, 209, 218 and 237, in particular at least one amino acid substitution selected from the group consisting of N121F, A194C, A209V, T218I, N237P and N237W, lead to an improved storage stability of this altered protease in washing and cleaning agents, in particular textile washing agents.

In preferred embodiments, the alterations according to the invention in at least one of the positions corresponding to positions 121, 194, 209, 218 and 237, in particular at least one amino acid substitution selected from the group consisting of N121F, A194C, A209V, T218I, N237P and N237W, lead to an improved storage stability of this altered protease in washing and cleaning agents, in particular textile washing agents, when the washing and cleaning agent, in particular textile washing agent, is stored for at least 1 week, 2 weeks, 3 weeks or 4 weeks at a temperature of 20° C., 25° C., 30° C. or 40° C.

In preferred embodiments, the alterations according to the invention in at least one of the positions corresponding to positions 121, 194, 209, 218 and 237, in particular at least one amino acid substitution selected from the group consisting of N121F, A194C, A209V, T218I, N237P and N237W, lead to an improved storage stability of this altered protease in washing and cleaning agents, in particular textile washing agents, when the storage stability is determined as described in example 1.

This is surprising in particular in that no protease with such alterations has been described so far for use in washing and cleaning agents, in particular textile washing agents. In particular, such proteases altered according to the invention have not been described in the context of an improved storage stability.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least two amino acid substitutions selected from the group consisting of N121F, A194C, A209V, T218I, N237P and N237W in at least two of the positions corresponding to positions 121, 194, 209, 218 and 237.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least three amino acid substitutions selected from the group consisting of N121F, A194C, A209V, T218I, N237P and N237W in at least three of the positions corresponding to positions 121, 194, 209, 218 and 237.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least four amino acid substitutions selected from the group consisting of N121F, A194C, A209V, T218I, N237P and N237W in at least four of the positions corresponding to positions 121, 194, 209, 218 and 237.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least five amino acid substitutions selected from the group consisting of N121F, A194C, A209V, T218I, N237P and N237W in at least five of the positions corresponding to positions 121, 194, 209, 218 and 237.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length and, in each case based on the numbering in accordance with SEQ ID NO:1, has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P.

In particularly preferred embodiments, the protease in accordance with the invention comprises an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the numbering is in each case based on the numbering in accordance with SEQ ID NO:1, and wherein the protease comprises no further changes besides the amino acid substitutions mentioned.

In preferred embodiments, the amino acid substitutions according to the invention described herein lead to improved storage stability of this altered protease in washing and cleaning agents, in particular textile washing agents. Proteases in accordance with the invention consequently have a higher residual activity after storage in washing and cleaning agents, in particular textile washing agents, than a reference protease, in particular wild-type protease in accordance with SEQ ID NO:1, and consequently allow improved removal of at least one, preferably multiple, protease-sensitive stains on textiles and/or hard surfaces, in particular dishes, after storage in washing and cleaning agents, in particular textile washing agents. Typical protease-sensitive stains comprise stains containing, e.g., egg (yolk), blood, milk and other proteins. Particularly preferably, proteases according to the invention allow improved removal of egg (yolk)-containing stains on textiles and/or hard surfaces, in particular dishes. An improvement in accordance with the invention in cleaning performance, in particular in proteolytic cleaning performance, is present when the protease exhibits an improved cleaning performance, relative to a reference protease, in particular wild-type protease in accordance with SEQ ID NO:1, preferably the cleaning performance after storage, on at least one protease-sensitive stain, which is preferably selected from the group consisting of blood-, egg (yolk)-, milk- and other protein-containing stains. An increase in stability during storage and/or during use, e.g., during the washing process, results in the enzymatic activity lasting longer and thus the cleaning performance being improved.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237, wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least two amino acid substitutions selected from the group consisting of N121F, A194C, A209V, T218I, N237P and N237W in at least two of the positions corresponding to positions 121, 194, 209, 218 and 237, wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least three amino acid substitutions selected from the group consisting of N121F, A194C, A209V, T218I, N237P and N237W in at least three of the positions corresponding to positions 121, 194, 209, 218 and 237, wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least four amino acid substitutions selected from the group consisting of N121F, A194C, A209V, T218I, N237P and N237W in at least four of the positions corresponding to positions 121, 194, 209, 218 and 237, wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least five amino acid substitutions selected from the group consisting of N121F, A194C, A209V, T218I, N237P and N237W in at least five of the positions corresponding to positions 121, 194, 209, 218 and 237, wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the protease shows improved storage stability in comparison with a reference protease (in particular wild-type protease in accordance with SEQ ID NO:1).

In preferred embodiments, the protease in accordance with the invention is a protease that exhibits proteolytic activity and has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the numbering is in each case based on the numbering in accordance with SEQ ID NO:1, wherein the protease comprises no further changes besides the amino acid substitutions mentioned, and wherein the protease shows improved storage stability in comparison with a reference protease (in particular wild-type protease in accordance with SEQ ID NO:1).

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237, wherein the protease shows improved storage stability in comparison with a reference protease (in particular wild-type protease in accordance with SEQ ID NO:1), when the washing and cleaning agent, in particular textile washing agent is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least two amino acid substitutions selected from the group consisting of N121F, A194C, A209V, T218I, N237P and N237W in at least two of the positions corresponding to positions 121, 194, 209, 218 and 237, wherein the protease shows improved storage stability in comparison with a reference protease (in particular wild-type protease in accordance with SEQ ID NO:1), when the washing and cleaning agent, in particular textile washing agent is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least three amino acid substitutions selected from the group consisting of N121F, A194C, A209V, T218I, N237P and N237W in at least three of the positions corresponding to positions 121, 194, 209, 218 and 237, wherein the protease shows improved storage stability in comparison with a reference protease (in particular wild-type protease in accordance with SEQ ID NO:1), when the washing and cleaning agent, in particular textile washing agent is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least four amino acid substitutions selected from the group consisting of N121F, A194C, A209V, T218I, N237P and N237W in at least four of the positions corresponding to positions 121, 194, 209, 218 and 237, wherein the protease shows improved storage stability in comparison with a reference protease (in particular wild-type protease in accordance with SEQ ID NO:1), when the washing and cleaning agent, in particular textile washing agent is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least five amino acid substitutions selected from the group consisting of N121F, A194C, A209V, T218I, N237P and N237W in at least five of the positions corresponding to positions 121, 194, 209, 218 and 237, wherein the protease shows improved storage stability in comparison with a reference protease (in particular wild-type protease in accordance with SEQ ID NO:1), when the washing and cleaning agent, in particular textile washing agent is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the protease shows improved storage stability in comparison with a reference protease (in particular wild-type protease in accordance with SEQ ID NO:1) when the washing and cleaning agent, in particular textile washing agent, is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the numbering is in each case based on the numbering in accordance with SEQ ID NO:1, wherein the protease comprises no further changes besides the amino acid substitutions mentioned, and wherein the protease shows improved storage stability in comparison with a reference protease (in particular wild-type protease in accordance with SEQ ID NO:1) when the washing and cleaning agent, in particular textile washing agent, is stored for at least 1 week, 2 weeks, 3 weeks or 4 weeks at a temperature of 20° C., 25° C., 30° C. or 40° C.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least two amino acid substitutions selected from the group consisting of N121F, A194C, A209V, T218I, N237P and N237W in at least two of the positions corresponding to positions 121, 194, 209, 218 and 237, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least three amino acid substitutions selected from the group consisting of N121F, A194C, A209V, T218I, N237P and N237W in at least three of the positions corresponding to positions 121, 194, 209, 218 and 237, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least four amino acid substitutions selected from the group consisting of N121F, A194C, A209V, T218I, N237P and N237W in at least four of the positions corresponding to positions 121, 194, 209, 218 and 237, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has at least five amino acid substitutions selected from the group consisting of N121F, A194C, A209V, T218I, N237P and N237W in at least five of the positions corresponding to positions 121, 194, 209, 218 and 237, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and comprises an amino acid sequence that is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99% identical to the amino acid sequence indicated in SEQ ID NO:1 over its entire length, wherein the protease, in each case based on the numbering in accordance with SEQ ID NO:1, has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the protease shows improved storage stability in comparison with a reference protease (in particular wild-type protease in accordance with SEQ ID NO:1) when the storage stability is determined as described in example 1.

In preferred embodiments, the protease in accordance with the invention is a protease that exhibits proteolytic activity and has an amino acid substitution combination selected from the group consisting of (i) A209V-N237P, (ii) A209V, (iii) N121F-A209V-N237P, (iv) A194C-N218I-N237W, (v) A209V-N237W and (vi) A209V-N218I-N237P, wherein the numbering is in each case based on the numbering in accordance with SEQ ID NO: 1, wherein the protease comprises no further changes besides the amino acid substitutions mentioned, and wherein the protease shows improved storage stability in comparison with a reference protease (in particular wild-type protease in accordance with SEQ ID NO:1) when the storage stability is determined as described in example 1.

Proteases according to the invention have a catalytic activity in washing and/or cleaning agents. In various embodiments, the proteases according to the invention can exhibit a proteolytic activity that is at least 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130% or more based on the wild type (SEQ ID NO:1).

The proteases in accordance with the invention have increased stability in washing and cleaning agents, in particular textile washing agents, in comparison with a reference protease (in particular wild-type protease in accordance with SEQ ID NO:1), e.g., to surfactants and/or bleaching agents and/or chelators, and/or to temperature influences, in particular to high temperatures, and/or to acidic or alkaline conditions and/or to pH alterations and/or to denaturing or oxidizing agents and/or to (auto)proteolytic degradation and/or to an alteration in the redox ratios. Protease variants that are more temperature-stable are therefore provided by particularly preferred embodiments of the invention.

Cleaning performance within the scope of the invention means the lightening performance on one or more stain(s), in particular on textiles, laundry or dishes. Within the scope of the invention, both the washing and/or cleaning agent comprising the protease, or the washing or cleaning liquor formed by said agent, and the protease itself have a particular cleaning performance. The cleaning performance of the enzyme thus contributes to the cleaning performance of the agent, or of the washing or cleaning liquor formed by the agent. The cleaning performance is preferably ascertained as described hereafter.

Washing or cleaning liquor is understood to mean the ready-to-use solution that contains the washing or cleaning agent and acts on textiles or fabric or hard surfaces, in particular dishes, and thus comes into contact with the stains present on textiles or fabrics or hard surfaces, in particular dishes. The washing or cleaning liquor is usually created when the washing or cleaning process begins and the washing or cleaning agent is diluted with water, e.g., in a dishwasher, a washing machine or in another suitable container.

The cleaning performance on textiles or fabrics can be determined in a washing system containing a washing agent in a dosage between 2.0 and 8.0 grams per liter of washing liquor.

The concentration of the peptide according to the invention in the washing agent intended for this washing system is 1×10⁻⁸ to 5 wt. %, preferably 0.0001 to 1 wt. %, more preferably 0.0005 to 0.5 wt. %, particularly preferably 0.001 to 0.1 wt. %, based on active protein and total weight of the washing agent.

The protein concentration can be determined using known methods, e.g., the BCA method (bicinchoninic acid; 2,2′-bichinolyl-4,4′-dicarboxylic acid) or the Biuret method (Gornall et al. J. Biol. Chem., 1948, 177, 751-766). In this regard, the active protein concentration can be determined via titration of the active centers using a suitable irreversible inhibitor and determination of the residual activity (Bender et al., J. Am. Chem. Soc., 1966, 88, 24, 5890-5913).

A liquid reference washing agent for such a washing system may be composed, for example, as follows (all figures in wt. %): 4.4% alkyl benzene sulfonic acid, 5.6% further anionic surfactants, 2.4% C₁₂₋₁₈ Na salts of fatty acids (soaps), 4.4% non-ionic surfactants, 0.2% phosphonates, 1.4% citric acid, 0.95% NaOH, 0.01% defoamer, 2% glycerol, 0.08% preservatives, 1% ethanol, and the remainder being demineralized water. The dosage of the liquid washing agent is preferably between 3.0 and 6.0 grams per liter of washing liquor, e.g., 3.0, 3.2, 3.5, 3.7, 4.0, 4.5, 4.7, 4.9 or 5.9 grams per liter of washing liquor. The washing process preferably takes place in a pH range between pH 7 and pH 10.5, preferably between pH 8 and pH 9.

The cleaning performance against stains on textiles is determined by measuring the degree of cleaning of the washed textiles. For example, the washing process can take place for 60 minutes at a temperature of 40° C. and the water can have a water hardness between 15.5° dH and 16.5° dH (German hardness).

The degree of whiteness, i.e., the lightening of the stains, as a measure of the cleaning performance is determined using optical measuring methods, preferably photometrically. A suitable device for this purpose is, for example, the Minolta CM508d spectrometer. Usually, the devices used for measurement are calibrated beforehand using a white standard, preferably a supplied white standard.

The activity-equivalent use of the relevant enzyme ensures that the respective enzymatic properties, for example the cleaning performance on certain stains, are compared even if the ratio of active substance to total protein (the values of the specific activity) diverges. In general, a low specific activity can be compensated by adding a larger amount of protein. Furthermore, the enzymes to be examined can also be used in the same amount of substance or amount by weight if the enzymes to be examined have a different affinity for the test substrate in an activity test. The expression “same amount of substance” in this context relates to a molar use of the enzymes to be examined. The expression “equal weight” relates to the use of the same weight of the enzymes to be examined.

Preferred embodiments of uses and agents according to the invention achieve such advantageous cleaning performance even at low temperatures, preferably in a temperature range of about 10° C. to about 60° C., preferably about 15° C. to about 40° C., particularly preferably about 20° C. to about 30° C.

The proteases according to the invention exhibit enzymatic activity, i.e., they are capable of hydrolyzing peptides and proteins, in particular in a washing or cleaning agent, preferably textile washing agent. A protease according to the invention is therefore an enzyme that catalyzes the hydrolysis of amide/peptide bonds in protein/peptide substrates and is thereby capable of cleaving proteins or peptides.

Methods for determining protease activity are familiar to and routinely used by a person skilled in the art in the field of enzyme technology. For example, such methods are disclosed in Tenside [Surfactants], volume 7 (1970), pp. 125-132. Alternatively, the protease activity can be determined via the release of the chromophore para-nitroaniline (pNA) from the substrate suc-L-Ala-L-Ala-L-Pro-L-Phe-p-nitroanilide (AAPF). The protease cleaves the substrate and releases pNA. The release of the pNA causes an increase in absorbance at 410 nm, the time profile of which is a measure of the enzymatic activity (cf. Del Mar et al., 1979). The measurement is carried out at a temperature of 25° C., a pH of 8.6 and a wavelength of 410 nm. The measurement time is 5 min and the measurement interval is 20 s to 60 s. The protease activity is usually expressed in protease units (PU). Suitable protease activities are 2.25, 5 or 10 PU per ml of washing liquor, for example. However, protease activity is not equal to zero.

An alternative test for determining the proteolytic activity of the proteases according to the invention is an optical measurement method, preferably a photometric method. The test suitable for this purpose comprises protease-dependent cleavage of the substrate protein casein. The casein is cleaved by the protease into a plurality of smaller partial products. The entirety of these partial products has an increased absorption at 290 nm with respect to non-cleaved casein, wherein this increased absorption can be determined using a photometer, and thus a conclusion can be drawn regarding the enzymatic activity of the protease.

Furthermore, a protease according to the invention is preferably a mature protease, i.e., the catalytically active molecule without a signal peptide/signal peptides and/or a propeptide/propeptides. Unless otherwise stated, the sequences indicated also refer to mature (processed) enzymes in each case.

In various embodiments of the invention, the protease is a free enzyme. This means that the protease can act directly with all components of an agent and, if the agent is a liquid agent, the protease is directly in contact with the solvent of the agent (e.g., water). In other embodiments, an agent may contain proteases that form an interaction complex with other molecules or that contain a “coating.” In this case, one or more protease molecule(s) can be separated from the other constituents of the agent by a structure surrounding them. Such a separating structure can arise due to, but is not limited to, vesicles, such as a micelle or a liposome. However, the surrounding structure may also be a virus particle, a bacterial cell or a eukaryotic cell. In various embodiments, an agent may contain cells of, for example, Bacillus pumilus or Bacillus subtilis or other expression strains that express the protease according to the invention, or cell culture supernatants of such cells.

The identity of nucleic acid or amino acid sequences is determined by a sequence comparison. This sequence comparison is based on the BLAST algorithm established and commonly used in the prior art (cf e.g., Altschul et al. (1990) “Basic local alignment search tool,” J. Mol. Biol., 215:403-410, and Altschul et al. (1997): “Gapped BLAST and PSI-BLAST: a new generation of protein database search programs,” Nucleic Acids Res., 25:3389-3402) and occurs in principle by similar sequences of nucleotides or amino acids in the nucleic acid or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. The Clustal series (cf. e.g., Chenna et al. (2003): “Multiple sequence alignment with the Clustal series of programs,” Nucleic Acid Res. 31:3497-3500), T-Coffee (cf. e.g., Notredame et al. (2000): “T-Coffee: A novel method for multiple sequence alignments,” J. Mol. Biol., 302:205-217) or programs based on these programs or algorithms, for example, are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid functional groups at the same positions or positions corresponding to one another in an alignment. In the case of amino acid sequences, the broader concept of homology takes conserved amino acid exchanges into account, i.e., amino acids having similar chemical activity, because these usually perform similar chemical activities within the protein. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above. Furthermore, the assignment of the positions is based on the mature protein. This assignment is also to be used in particular when the amino acid sequence of a protease according to the invention comprises a higher number of amino acid radicals than the protease according to SEQ ID NO:1. Proceeding from the stated positions in the amino acid sequence of the protease according to SEQ ID NO:1, the alteration positions in a protease according to the invention are those which are assigned to said positions in an alignment.

In addition to the amino acid alterations explained above, proteases according to the invention can have further amino acid alterations, in particular amino acid substitutions, insertions or deletions. Such proteases are, for example, developed by targeted genetic modification, i.e., by mutagenesis methods, and optimized for specific applications or with regard to specific properties (e.g., with regard to their catalytic activity, stability, etc.). Furthermore, nucleic acids according to the invention can be introduced into recombination approaches and thus used to produce completely novel proteases or other polypeptides. The aim is to introduce targeted mutations such as substitutions, insertions or deletions into the known molecules in order, e.g., to improve the cleaning performance of enzymes according to the invention. For this purpose, in particular the surface charges and/or the isoelectric point of the molecules and thus their interactions with the substrate can be altered. For instance, the net charge of the enzymes can be altered in order to influence the substrate binding, in particular for use in washing and cleaning agents. Alternatively or additionally, the stability or catalytic activity of the protease can be increased by one or more corresponding mutations and its cleaning performance can thereby be improved. Advantageous properties of individual mutations, e.g., individual substitutions, can complement one another. A protease that has already been optimized with regard to specific properties, for example with regard to its stability on storage and/or activity and/or its tolerance in relation to the substrate spectrum, can therefore also be developed within the scope of the invention.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a A is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

The invention therefore also relates to a protease which is characterized in that it is obtained from a protease as described herein as the starting molecule by single or multiple conservative amino acid substitution, the protease in the numbering according to SEQ ID NO:1 having at least one of the above-described amino acid substitutions. The term “conservative amino acid substitution” means the exchange (substitution) of one amino acid functional group for another amino acid functional group, with this exchange not resulting in a change to the polarity or charge at the position of the exchanged amino acid, e.g., the exchange of a nonpolar amino acid functional group for another nonpolar amino acid functional group. Conservative amino acid substitutions within the context of the invention include, for example: G=A=S, I=V=L=M, D=E, N=Q, K=R, Y=F, S=T, G=A=I=V=L=M=Y=F=W=P=S=T.

Alternatively or additionally, the protease is characterized in that it is obtained from a protease in accordance with the invention as starting molecule by fragmentation or deletion, insertion or substitution mutagenesis and comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease, in each case based on the numbering in accordance with SEQ ID NO: 1, has at least one amino acid substitution selected from the group consisting of N121F, A194C, A209V, T218I, N237P and N237W in at least one of the positions corresponding to positions 121, 194, 209, 218 and 237.

For instance, it is possible to delete individual amino acids at the termini or in the loops of the enzyme without the proteolytic activity being lost or diminished in the process. Furthermore, such fragmentation, deletion mutagenesis, insertion mutagenesis or substitution mutagenesis can also, for example, reduce the allergenicity of the enzymes in question and thus improve their overall applicability. Advantageously, the enzymes retain their proteolytic activity even after mutagenesis, i.e., their proteolytic activity corresponds at least to that of the starting enzyme, i.e., in a preferred embodiment, the proteolytic activity is at least 100%, preferably at least 105%, more preferably at least 110%, even more preferably at least 120% or more, of the activity of the starting enzyme. Further substitutions can also demonstrate advantageous effects. Both individual and multiple contiguous amino acids can be replaced with other amino acids.

Advantageous positions for sequence alterations, in particular substitutions, of the protease according to SEQ ID NO:1, which are preferably of significance when transferred to homologous positions of the proteases according to the invention and which impart advantageous functional properties to the protease, are therefore the positions which correspond to the positions described herein in an alignment, i.e., in the numbering according to SEQ ID NO:1. The following amino acid functional groups are located in the wild-type protease molecule at the positions mentioned: 121N, 194A, 209A, 218T, 237N.

Further confirmation of the correct assignment of the amino acids to be altered, i.e., in particular their functional correspondence, can be provided by comparative tests, based on which the two positions assigned to one another on the basis of an alignment in the two proteases compared with one another are altered in the same way and observation is carried out to determine whether the enzymatic activity is altered in the same way in the two proteases. If, for example, an amino acid exchange in a specific position of the protease in accordance with SEQ ID NO:1 is accompanied by an alteration of an enzymatic parameter, e.g., an increase in the K_{M} value, and a corresponding alteration of the enzymatic parameter, e.g., likewise an increase in the K_{M} value, is observed in a protease variant in accordance with the invention of which the amino acid exchange has been achieved by the same introduced amino acid, this can therefore be considered to be confirmation of the correct assignment.

All of these aspects are also applicable to the methods according to the invention for producing a protease.

A method in accordance with the invention for producing a protease comprises introducing at least one amino acid substitution in at least one of the positions corresponding to positions 121, 194, 209, 218 and 237, based on the numbering in accordance with SEQ ID NO:1, selected from the group consisting of N121F, A194C, A209V, T218I, N237P and N237W, into a starting molecule that has an amino acid sequence that has at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% or 100% sequence identity with the amino acid sequence indicated in SEQ ID NO:1 over its entire length.

A method according to the invention can also comprise one or more of the following method steps:
(a) introducing a single or multiple conservative amino acid substitution, wherein the protease, in each case based on the numbering in accordance with SEQ ID NO:1, has at least one amino acid substitution selected from the group consisting of N121F, A194C, A209V, T218I, N237P and N237W in at least one of the positions corresponding to positions 121, 194, 209, 218 and 237;
(b) altering the amino acid sequence by fragmentation or deletion, insertion or substitution mutagenesis such that the protease comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease, in each case based on the numbering in accordance with SEQ ID NO: 1, has at least one amino acid substitution selected from the group consisting of N121F, A194C, A209V, T218I, N237P and N237W in at least one of the positions corresponding to positions 121, 194, 209, 218 and 237.

All embodiments also apply to the methods according to the invention.

In a further embodiment of the invention, a previously described protease is further stabilized. In principle, all stabilization options described and/or expedient in the prior art are conceivable. Preference is given to those stabilizations which are achieved via mutations of the enzyme itself because such stabilizations do not require any further working steps after the recovery of the enzyme. Examples of sequence alterations suitable for this purpose are specified above. Further suitable sequence alterations are known from the prior art.

Further possibilities for stabilization are, for example:
altering the binding of metal ions, in particular the calcium binding sites, for example, by exchanging one or more of the amino acid(s) involved in the calcium binding for one or more negatively charged amino acids and/or by introducing sequence alterations in at least one of the sequences of the two amino acids arginine/glycine;
protecting against the influence of denaturing agents such as surfactants by mutations which cause an alteration of the amino acid sequence on or at the surface of the protein;
exchanging amino acids that are close to the N-terminus for those that presumably come into contact with the rest of the molecule via non-covalent interactions and thus contribute to maintaining the globular structure.

Preferred embodiments are those in which the enzyme is stabilized in a plurality of ways because a plurality of stabilizing mutations act additively or synergistically.

Alternatively or additionally, the protease according to the invention can be combined with at least one reversible inhibitor compound selected from the group consisting of peptide inhibitors, in particular peptide aldehydes, polyols, in particular glycerol and 1,2-propylene glycol, benzamidine hydrochloride, borax, boric acids, boronic acids or the salts or esters or derivatives thereof, in particular phenylboronic acid derivatives or 4-formylphenylboronic acid (4-FPBA), in order to further increase the stability of the protease in washing and cleaning agents. Particularly preferred reversible protease inhibitors include boric acid, 4-FPBA and peptide inhibitors.

In various embodiments, the enzyme and the inhibitor compound can be pre-formulated in an enzyme composition. Protease preparations that are preferably used contain between 0.1 and 40 wt. %, preferably between 0.2 and 30 wt. %, particularly preferably between 0.4 and 20 wt. %, and in particular between 0.8 and 10 wt. %, of the enzyme protein. In such compositions, the inhibitor compound can be present in an amount of from 0.05 to 35 wt. %, preferably from 0.05 to 10 wt. %, based on the total weight in the enzyme composition. This enzyme composition, which is also a constituent of the present invention, can then be used in washing or cleaning agents, specifically in amounts which lead to the desired final concentrations in the washing or cleaning agent.

A further subject matter of the invention is a protease as described herein, characterized in that it has at least one chemical modification. A protease having such an alteration is referred to as a derivative, i.e., the protease is derivatized. In the context of the present application, derivatives are thus understood to mean proteins whose pure amino acid chain has been chemically modified. Such derivatizations can be achieved, for example, in vivo by the host cell that expresses the protein. In this regard, couplings of low-molecular-weight compounds such as lipids or oligosaccharides are particularly noteworthy. Derivatizations can also be made in vitro, for instance by means of chemical conversion of a side chain of an amino acid or by means of covalent bonding of another compound to the protein. For example, it is possible to couple amines to carboxyl groups of an enzyme in order to alter the isoelectric point. Another such compound may also be another protein that is bound to a protein according to the invention via bifunctional chemical compounds, for example. Derivatization is likewise understood to mean covalent bonding to a macromolecular carrier or a non-covalent inclusion in suitable macromolecular cage structures. Derivatizations may, for example, affect the substrate specificity or bonding strength to the substrate or cause a temporary blockage of the enzymatic activity if the coupled substance is an inhibitor. This can be expedient, for example, for the period of storage. Such modifications may further affect the stability or enzymatic activity. They can also be used to reduce the allergenicity and/or immunogenicity of the protein and for example increase its skin compatibility. For example, couplings with macromolecular compounds, for example polyethylene glycol, can improve the protein in terms of stability and/or skin compatibility. Derivatives of a protein according to the invention can also be understood in the broadest sense to be preparations of these proteins. A protein can, depending on the recovery, processing or preparation thereof, be combined with various other substances, e.g., from the culture of the producing microorganisms. A protein may also have been deliberately mixed with other substances, e.g., to increase its storage stability. Therefore, all preparations of a protein according to the invention are also in accordance with the invention. This is also independent of whether or not it actually exhibits this enzymatic activity in a particular preparation. This is because it may be desirable for it to have no activity or only a small amount of activity during storage and to only exhibit its enzymatic function at the time of use. This can be controlled via appropriate accompanying substances, for example. In particular, the joint preparation of proteases with specific inhibitors is possible in this regard. Within the scope of the present invention, of all the proteases or protease variants and/or derivatives described herein, those with a storage stability and/or catalytic activity and/or substrate tolerance and/or cleaning performance that is improved relative to the starting variant are particularly preferred, wherein the catalytic activity and/or cleaning performance is determined as described herein.

A further subject matter of invention is a nucleic acid coding for a protease according to the invention, and a vector containing such a nucleic acid, in particular a cloning vector or an expression vector. These can be DNA or RNA molecules. They can be present as a single strand, as a single strand complementary to said single strand or as a double strand. In particular in the case of DNA molecules, the sequences of the two complementary strands must be taken into account in all three possible reading frames. Furthermore, it must be taken into account that different codons, i.e., base triplets, can code for the same amino acids such that a certain amino acid sequence can be coded by a plurality of different nucleic acids. Due to this degeneracy of the genetic code, all of the nucleic acid sequences which can code any of the proteases described above are included in this subject matter of the invention. A person skilled in the art is able to determine these nucleic acid sequences beyond a doubt because, despite the degeneracy of the genetic code, defined amino acids can be assigned to individual codons. Therefore, a person skilled in the art proceeding from said amino acid sequence can easily determine nucleic acids coding for said amino acid sequence. Furthermore, in the case of nucleic acids according to the invention, one or more codons can be replaced by synonymous codons. This aspect relates in particular to the heterologous expression of the enzymes according to the invention. Thus, each organism, for example a host cell of a production strain, has a certain codon usage. “Codon usage” is understood to mean the translation of the genetic code into amino acids by the relevant organism. Bottlenecks can occur in protein biosynthesis if the codons on the nucleic acid in the organism are faced with a comparatively small number of loaded tRNA molecules. Although coding for the same amino acid, this results in a codon being translated less efficiently in the organism than a synonymous codon coding for the same amino acid. Due to the presence of a higher number of tRNA molecules for the synonymous codon, this can be translated more efficiently in the organism.

It is possible for a person skilled in the art to use methods which are currently generally known, e.g., chemical synthesis or polymerase chain reaction (PCR), in conjunction with molecular biological and/or protein-chemical standard methods, to produce the corresponding nucleic acids and even complete genes on the basis of known DNA and/or amino acid sequences. Such methods are known, e.g., from Sambrook, J., Fritsch, E. F. and Maniatis, T. 2001. Molecular cloning: a laboratory manual, 3. Edition Cold Spring Laboratory Press.

For the purposes of the present invention, “vectors” are understood to mean elements consisting of nucleic acids that contain a nucleic acid according to the invention as the characteristic nucleic acid region. They are able to establish these as a stable genetic element in a species or cell line over several generations or cell divisions. Vectors are, in particular when used in bacteria, special plasmids, i.e., circular genetic elements. In the context of the present invention, a nucleic acid according to the invention is cloned into a vector. The vectors include, for example, those originating from bacterial plasmids, viruses or bacteriophages, or predominantly synthetic vectors or plasmids having elements of a wide variety of origins. With the other genetic elements present in each case, vectors are able to establish themselves as stable units in the particular host cells over several generations. They can be present extrachromosomally as separate units or can be integrated into a chromosome or chromosomal DNA. Expression vectors comprise nucleic acid sequences that allow them to replicate in the host cells containing them, preferably microorganisms, particularly preferably bacteria, and to express a contained nucleic acid there. The expression is influenced in particular by the promoter or promoters that regulate transcription. In principle, the expression can take place by the natural promoter originally located before the nucleic acid to be expressed, but also by a promoter of the host cell provided on the expression vector or also by a modified or completely different promoter of another organism or another host cell. In the present case, at least one promoter is provided for the expression of a nucleic acid according to the invention and used for the expression thereof. Expression vectors can also be regulatable, for example by changing the cultivation conditions or when a certain cell density of the host cells containing them is reached or by adding certain substances, in particular activators of gene expression. An example of such a substance is the galactose derivative isopropyl β-D-thiogalactopyranoside (IPTG), which is used as an activator of the bacterial lactose operon (lac operon). In contrast to expression vectors, the nucleic acid contained is not expressed in cloning vectors.

The invention further relates to a non-human host cell containing a nucleic acid according to the invention or a vector according to the invention, or containing a protease according to the invention, in particular one that secretes the protease into the medium surrounding the host cell. Preferably, a nucleic acid according to the invention or a vector according to the invention is transformed into a microorganism that then represents a host cell according to the invention. Alternatively, individual components, i.e., nucleic acid parts or fragments of a nucleic acid according to the invention, can also be introduced into a host cell in such a way that the resulting host cell contains a nucleic acid according to the invention or a vector according to the invention. This procedure is particularly suitable when the host cell already contains one or more constituents of a nucleic acid according to the invention or a vector according to the invention and the further constituents are then supplemented accordingly. Methods for transforming cells are established in the prior art and are well known to a person skilled in the art. In principle all cells, i.e., prokaryotic or eukaryotic cells, are suitable as host cells. Host cells which can be managed in a genetically advantageous manner, for example with regard to transformation with the nucleic acid or the vector and its stable establishment, are preferred, for example single-cell fungi or bacteria. Furthermore, preferred host cells are distinguished by good microbiological and biotechnological manageability. This relates, for example, to easy cultivation, high growth rates, low requirements for fermentation media and good production and secretion rates for foreign proteins. Preferred host cells according to the invention secrete the (transgenically) expressed protein into the medium surrounding the host cells. Furthermore, the proteases can be modified by the cells producing them after their production, e.g., by linking sugar molecules, formylations, aminations, etc. Such post-translational modifications can functionally influence the protease.

Further preferred embodiments are host cells that can be regulated in their activity owing to genetic regulatory elements that are provided, for example, on the vector but can also be present in these cells from the outset. Expression in said cells may be induced, for example, by controlled addition of chemical compounds used as activators, by changing the cultivation conditions or when a particular cell density is reached. This allows economic production of the proteins according to the invention. An example of such a compound is IPTG, as described above.

Prokaryotic or bacterial cells are preferred host cells. Bacteria are characterized by short generation times and low demands on cultivation conditions. This makes it possible to establish cost-effective cultivation methods or production methods. In addition, a person skilled in the art will have a wealth of experience in the case of bacteria in fermentation technology. Gram-negative or gram-positive bacteria can be suitable for a specific production for many different reasons to be determined experimentally in each individual case, such as nutrient sources, product formation rate, time needed, etc. In gram-negative bacteria, such as Escherichia coli, a plurality of proteins are secreted into the periplasmic space, i.e., into the compartment between the two membranes enclosing the cells. This can be advantageous for specific applications. Furthermore, gram-negative bacteria can also be designed such that they discharge the expressed proteins not only into the periplasmic space but into the medium surrounding the bacterium. Gram-positive bacteria such as bacilli or actinomycetes or other representatives of actinomycetes in contrast do not have an outer membrane, such that secreted proteins are immediately released into the medium surrounding the bacteria, usually the nutrient medium, from which the expressed proteins can be purified. They can be isolated directly from the medium or further processed. In addition, gram-positive bacteria are related or identical to most origin organisms for technically important enzymes and usually themselves form comparable enzymes, such that they have a similar codon usage and their protein synthesis apparatus is naturally aligned accordingly. Host cells according to the invention may be altered in terms of their requirements for culture conditions, have different or additional selection markers, or also express different or additional proteins. In particular, this may also involve those host cells which express a plurality of proteins or enzymes. The present invention can be applied in principle to all microorganisms, in particular to all fermentable microorganisms, particularly preferably to those of the genus Bacillus, and allows proteins according to the invention to be produced using such microorganisms. Such microorganisms then represent host cells for the purposes of the invention. In a further embodiment of the invention, the host cell is characterized in that it is a bacterium, preferably one selected from the group of the genera of Escherichia, Klebsiella, Bacillus, Staphylococcus, Corynebacterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

The host cell may also be a eukaryotic cell, however, which is characterized in that it has a cell nucleus. The invention therefore further relates to a host cell that is characterized in that it has a nucleus. In contrast with prokaryotic cells, eukaryotic cells are capable of post-translationally modifying the protein formed. Examples thereof are fungi, such as actinomycetes or yeasts, such as Saccharomyces or Kluyveromyces. This can be particularly advantageous, for example, if the proteins are to undergo specific modifications in connection with their synthesis, which modifications make such systems possible. Modifications carried out by eukaryotic systems, in particular in connection with the protein synthesis, include, for example, the binding of low-molecular-weight compounds such as membrane anchors or oligosaccharides. Such oligosaccharide modifications can be desirable, for example, to reduce the allergenicity of an expressed protein. Coexpression with the enzymes naturally formed by such cells, such as cellulases, can also be advantageous. Furthermore, for example, thermophilic fungal expression systems can be particularly suitable for expression of temperature-resistant proteins or variants.

The host cells according to the invention are cultured and fermented in the usual manner, for example in discontinuous or continuous systems. In the first case, a suitable nutrient medium is inoculated with the host cells and the product is harvested from the medium after a period to be determined experimentally. Continuous fermentations are characterized by achieving a flow equilibrium in which cells partially die off over a comparatively long period but also grow back and, at the same time, the protein formed can be removed from the medium.

Host cells according to the invention are preferably used to produce proteases according to the invention. The invention therefore further relates to a method for producing a protease, comprising
(a) cultivating a host cell according to the invention, and
(b) isolating the protease from the culture medium or from the host cell.

This subject matter of the invention preferably comprises fermentation processes. Fermentation processes are known per se from the prior art and represent the actual large-scale production step, generally followed by a suitable purification method for the product produced, for example for the proteases according to the invention. All fermentation processes that are based on a corresponding method for producing a protease according to the invention represent embodiments of this subject matter of the invention. Fermentation processes that are characterized in that the fermentation is carried out via a feed strategy are considered in particular. In this case, the media constituents that are consumed by the continuous cultivation are added. As a result, considerable increases can be achieved both in the cell density and in the cell mass or dry mass and/or in particular in the activity of the protease of interest. Furthermore, the fermentation can also be designed such that unwanted metabolic products are filtered out or neutralized by adding buffers or suitable counterions. The prepared protease can be harvested from the fermentation medium. Such a fermentation process is preferred over isolation of the protease from the host cell, i.e., product preparation from the cell mass (dry mass), but requires the provision of suitable host cells or one or more suitable secretion markers or mechanisms and/or transport systems so that the host cells secrete the protease into the fermentation medium. Without secretion, the isolation of the protease from the host cell, i.e., purification thereof from the cell mass, can alternatively take place, for example, by means of precipitation with ammonium sulfate or ethanol or by means of chromatographic cleaning.

All of the above-mentioned aspects can be combined to form methods in order to produce the protease according to the invention.

A further subject of the invention is a washing and/or cleaning agent that is characterized in that it contains a protease according to the invention as described herein. Preferably, the agent is a textile washing agent, in particular a liquid textile washing agent.

In preferred embodiments, the protease according to the invention is used in agents that are substantially free from boron-containing compounds. “Substantially free from boron-containing compounds” in this context means that the corresponding agents contain less than 2 wt. %, preferably less than 1 wt. %, more preferably less than 0.5 wt. %, and particularly preferably less than 0.1 wt. %, of boron-containing compounds, based on the total weight of the agent. In very particularly preferred embodiments, such agents are free from boron-containing compounds, i.e., in particular they do not contain any boric acid and/or 4-FPBA.

In preferred embodiments, the protease according to the invention is used in agents that are substantially free from phosphonate-containing compounds. “Substantially free from phosphonate-containing compounds” in this context means that the corresponding agents contain less than 2 wt. %, preferably less than 1 wt. %, more preferably less than 0.5 wt. %, and particularly preferably less than 0.1 wt. %, of phosphonate-containing compounds, based on the total weight of the agent. In particularly preferred embodiments, these agents are free from phosphonate-containing compounds.

In preferred embodiments, the protease according to the invention is used in agents that are substantially free from phosphate-containing compounds. “Substantially free from phosphate-containing compounds” in this context means that the corresponding agents contain less than 2 wt. %, preferably less than 1 wt. %, more preferably less than 0.5 wt. %, and particularly preferably less than 0.1 wt. %, of phosphate-containing compounds, based on the total weight of the agent. In particularly preferred embodiments, these agents are free from phosphate-containing compounds.

According to the invention, all conceivable types of washing or cleaning agents are to be understood as washing or cleaning agents, both concentrates and undiluted agents, for use on a commercial scale, in washing machines or for hand washing or cleaning. These include, for example, washing agents for textiles, carpets or natural fibers, for which the term “washing agents” is used. These also include, for example, dishwashing detergents for dishwashers (automatic dishwasher detergents) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and automatic dishwashing detergents, also, for example, scouring agents, glass cleaners, WC rim blocks, etc. The washing and cleaning agents within the scope of the invention also include auxiliary washing agents that are added to the actual washing agent during manual or automatic textile washing in order to achieve a further effect. Furthermore, within the scope of the invention, washing and cleaning agents also include textile pre-treatment agents and post-treatment agents, e.g., agents with which the item of laundry is brought into contact before the actual washing, e.g., for dissolving stubborn soiling, and also agents which, in a step downstream of the actual textile washing, impart further desirable properties to the laundry item, such as a pleasant feel, crease resistance or low static charge. Inter alia, softeners are included in the latter agents. This also comprises agents for use in (semi-) automated washing or cleaning systems such as floor-mopping robots or wet vacuum cleaners.

Agents according to the invention, which may be in the form of powdered or granular solids, in compacted or re-compacted particulate form, homogeneous solutions or suspensions, may contain, in addition to a protease according to the invention, all known ingredients that are conventional in such agents, wherein preferably at least one further ingredient is present in the agent. Agents according to the invention can in particular contain surfactants, builders, polymers, glass corrosion inhibitors, corrosion inhibitors, bleaching agents such as peroxygen compounds, bleach activators or bleach catalysts. They may also contain water-miscible organic solvents, further enzymes, enzyme stabilizers, sequestering agents, electrolytes, pH regulators and/or further auxiliaries, such as optical brighteners, graying inhibitors, dye transfer inhibitors, foam regulators, as well as dyes and fragrances, and combinations thereof. Advantageous ingredients of agents according to the invention are disclosed in WO 2009/121725, starting at the penultimate paragraph of page 5 and ending after the second paragraph on page 13. Reference is expressly made to this disclosure and the disclosure therein is incorporated into the present patent application.

Agents according to the invention advantageously contain the protease according to the invention in an amount of 2 g to 20 mg, preferably of 5 g to 17.5 mg, particularly preferably of 20 g to 15 mg, and very particularly preferably of 50 g to 10 mg per g of the agent. In various embodiments, the concentration of the protease (active enzyme) described herein in the agent is >0 to 1 wt. %, preferably 0.0001 or 0.001 to 0.1 wt. %, based on the total weight of the agent.

Agents according to the invention contain the protease according to the invention increasingly preferably in an amount of 1×10⁻⁸ to 5 wt. %, of 0.0001 to 1 wt. %, of 0.0005 to 0.5 wt. %, of 0.001 to 0.1 wt. %, in each case based on active protein and based on the total weight of the agent.

Further embodiments comprise all solid, powdery, liquid, gel-like or pasty dosage forms of agents according to the invention, which may optionally also consist of a plurality of phases and may be present in compressed or non-compressed form. Agents according to the invention can be in the form of a free-flowing powder, in particular having a bulk density of 300 g/l to 1200 g/l, in particular 500 g/l to 900 g/l or 600 g/l to 850 g/l. The solid administration forms of agents according to the invention further include extrudates, granules, tablets or pouches. Alternatively, agents according to the invention can also be in a liquid, gel or pasty form, e.g., in the form of a non-aqueous liquid washing agent or a non-aqueous paste or in the form of an aqueous liquid washing agent or an aqueous paste. Liquid agents are generally preferred. Furthermore, agents according to the invention can be in the form of a single-component system. Such agents consist of one phase. Alternatively, agents according to the invention can also consist of multiple phases. Such an agent is accordingly divided into a plurality of components.

In a preferred embodiment, the agent according to the invention is a textile washing agent.

In a preferred embodiment, the agent according to the invention is a liquid textile washing agent.

In a preferred embodiment, the agent according to the invention is a pre-portioned washing agent, in particular a washing agent portion unit comprising a washing agent preparation according to the invention and a water-soluble film which completely encloses the washing agent preparation.

The water-soluble film in which the washing agent preparation is packaged can comprise one or more structurally different water-soluble polymer(s). Suitable water-soluble polymer(s) are in particular polymers from the group of (optionally acetalized) polyvinyl alcohols (PVAL) and copolymers thereof. Suitable water-soluble films for use are marketed, inter alia, by the company MonoSol LLC, for example, under the names M8630, M8720, M8310, C8400 or M8900. For example, films with the name Solublon® PT, Solublon® GA, Solublon® KC or Solublon® KL by Aicello Chemical Europe GmbH or the VF-HP films by Kuraray are also suitable.

If the agents according to the invention are in liquid form, they preferably contain more than 40 wt. %, preferably 50 to 90 wt. %, and particularly preferably 60 to 80 wt. %, of water, based on their total weight.

Agents according to the invention can contain only one protease according to the invention. Alternatively, they can also contain further hydrolytic enzymes or other enzymes in a concentration expedient for the effectiveness of the agent. A further embodiment of the invention is thus represented by agents that further comprise one or more further enzymes. Further enzymes which can preferably be used are all enzymes which can exhibit catalytic activity in the agent according to the invention, in particular a lipase, amylase, cellulase, hemicellulase, mannanase, tannase, xylanase, xanthanase, xytoglucanase, B-glucosidase, pectinase, carrageenase, perhydrolase, oxidase, oxidoreductase or another protease, which is different from the proteases according to the invention, as well as mixtures thereof. Additional enzymes are contained in the agent advantageously in an amount of from 1×10⁻⁸ to 5 wt. %, based on active protein. Increasingly preferably, each further enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Particularly preferably, the enzymes exhibit synergistic cleaning performance with respect to particular dirt or stains, i.e., the enzymes contained in the agent assist one another in their cleaning performance. Very particularly preferably, such a synergism exists between the protease present according to the invention and a further enzyme of an agent according to the invention. Synergistic effects can occur not only between different enzymes but also between one or more enzymes and other ingredients of the agent according to the invention.

Textile washing agents preferred according to the invention have at least one protease and at least one amylase. In a further preferred embodiment of the invention, textile washing agents have at least one protease and at least one cellulase. In a further preferred embodiment, textile washing agents have at least one protease, and at least one lipase. In a further preferred embodiment, textile washing agents have at least one protease, at least one amylase, and at least one lipase. In a further preferred embodiment, textile washing agents have at least one protease, at least one amylase, and at least one cellulase. In a further preferred embodiment, textile washing agents have at least one protease, at least one amylase, at least one cellulase, and at least one lipase. Textile washing agents which have 3 to 10 different enzymes are particularly preferred, it being possible for textile washing agents which have 3 to 10 different types of enzymes to be particularly preferred with regard to the cleaning performance over a very broad spectrum of stains.

In the cleaning agents described herein, the enzymes to be used can further be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These pre-packaged preparations include, for example, the solid preparations obtained through granulation, extrusion, or lyophilization or, in particular in the case of liquid or gel agents, solutions of the enzymes, which are advantageously maximally concentrated, have a low water content, and/or are supplemented with stabilizers or other auxiliaries.

Alternatively, the enzymes can also be encapsulated, for both the solid and the liquid administration form, e.g., by spray-drying or extrusion of the enzyme solution together with a preferably natural polymer or in the form of capsules, e.g., those in which the enzymes are enclosed in a set gel, or in those of the core-shell type, in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using methods that are known per se, for example by shaking or roll granulation or in fluidized bed processes. Advantageously, such granules are low in dust, for example due to the application of polymeric film-formers, and stable in storage due to the coating.

Furthermore, it is possible to formulate two or more enzymes together such that a single granule exhibits a plurality of enzyme activities.

The enzymes can also be introduced into water-soluble films, such as those used in the formulation of washing and cleaning agents in a unit dosage form. Such a film allows the enzymes to be released after contact with water. As used herein, “water-soluble” refers to a film structure that is preferably completely water-soluble. Preferably, such a film consists of (completely or partially hydrolyzed) polyvinyl alcohol (PVA).

A further subject of the invention is a method for cleaning textiles and/or hard surfaces, in particular dishes, which is characterized in that an agent containing a protease according to the invention is used in at least one method step. In various embodiments, the method described is characterized in that the protease is used at a temperature of approximately 0° C. to approximately 100° C., preferably approximately 20° C. to approximately 60° C. and more preferably approximately 20° C. to approximately 40° C.

This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent.

Because proteases according to the invention by nature already have hydrolytic activity and also exhibit said activity in media that otherwise have no cleaning power, such as in a simple buffer, an individual and/or the single step of such a method can consist of bringing a protease according to the invention into contact with the stain as the only cleaning-active component, preferably in a buffer solution or in water. This represents a further embodiment of this subject matter of the invention.

Alternative embodiments of this subject matter of the invention also include methods for treating textile raw materials or for textile care, in which a protease according to the invention is active in at least one method step. Among these, methods for textile raw materials, fibers or textiles comprising natural constituents are preferred, and very particularly for those comprising wool or silk.

A further subject of the invention relates to a method for removing protease-sensitive stains, in particular egg (yolk)-containing stains, from textiles and/or hard surfaces, in particular dishes, wherein, in at least one method step, an agent containing a protease according to the invention as described herein and/or a protease according to the invention as described herein is used.

Finally, the invention also encompasses the use of the proteases described herein in washing or cleaning agents, for example as described above, for the (improved) removal of peptide- or protein-containing stains, for example from textiles and/or hard surfaces.

In a further preferred embodiment, the invention relates to the use of a protease according to the invention described herein in a washing or cleaning agent, in particular textile washing agent, to improve the storage stability of a protease in such a protease-containing washing or cleaning agent, in particular textile washing agent.

In a further preferred embodiment, the invention relates to the use of a protease according to the invention described herein in a washing or cleaning agent, in particular a textile washing agent, for the removal of at least one protease-sensitive stain.

All aspects, subjects, and embodiments described for the protease according to the invention and agents containing them are also applicable to these subjects of the invention. Therefore, reference is expressly made at this point to the disclosure at the corresponding point where it is indicated that this disclosure also applies to the methods and uses according to the invention.

### EXAMPLES

TABLE 1
CLEANING AGENT MATRIX USED

| | Wt. % of active substance |
|---|---|
| Chemical name | in formulation |
| Alkyl benzene sulfonic acid | 22 |
| Non-ionic surfactants | 25 |
| C12-14 fatty acid | 7 |
| 1,2-propanediol | 4 |
| Glycerol | 10 |
| Monoethanolamine | 6 |
| Phosphonate | 1 |
| Other (cleaning-active polymers, | minors |
| preservatives, perfume, etc.) | |
| Water | To make up to 100 |
| Without enzymes, pH 7.5 | |

TABLE 2
PROTEASES USED

| | Amino acid substitutions relative to SEQ ID NO: 1, |
|---|---|
| | counting in accordance with SEQ ID NO: 1 |
| Protease 1 (P1) | Protease in accordance with SEQ ID NO: 1 (wild type) |
| Protease 2 (P2) | A209V-N237P |
| Protease 3 (P3) | A209V |
| Protease 4 (P4) | N121F-A209V-N237P |
| Protease 5 (P5) | A194C-T218I-N237W |
| Protease 6 (P6) | A209V-N237W |
| Protease 7 (P7) | A209V-N218I-N237P |

### Example 1: Determining the Storage Stability

The storage stability of the proteases in accordance with table 2 was determined in a washing agent composition in accordance with table 1 with Bacillus subtilis culture supernatants containing the protease to be investigated. The culture supernatants were diluted 1:5 with a washing agent solution (1:1 washing agent matrix and deionized water). The washing agent solution containing the protease to be investigated was stored at a temperature of 30° C. for 2 weeks.

The activity of the protease to be investigated was measured immediately after preparation of the test mixtures and after 2 weeks of storage.

### Protease Activity Assay

The activity of the protease is determined by releasing the chromophore para-nitroaniline from the substrate succinyl alanine-alanine-proline-phenylalanine-para-nitroanilide (AAPF-pNA; Bachem L-1400). The release of the pNA causes an increase in extinction at 410 nm, the time profile of which is a measure of the enzymatic activity.

The measurement was carried out at a temperature of 30° C., a pH of 8.6 and a wavelength of 410 nm. The measurement time was 5 min at a measuring interval of 20 to 60 seconds.

### Measurement Approach:

10 μL AAPF solution (70 mg/mL)
1000 μL Tris/HCl (0.1 M, pH 8.6 with 0.1% Brij 35)
10 μL diluted protease solution
Kinetics produced over 5 min at 25° C. (410 nm)

The protease activity before storage was normalized to 100%. The difference values of the residual activity (RA) of the protease of each batch after storage are indicated below in comparison with the initial value before storage. A difference of ≥5% is considered significant.

RA (14 d)

| Name | Average | Stdevp (n > 2) |
|---|---|---|
| Protease 1 (P1) WT | 4% | 1.5% |
| Protease 1 (P2) | 12% | 1.0% |
| Protease 1 (P3) | 10% | 1.2% |
| Protease 1 (P4) | 38% | 0.0% |
| Protease 1 (P5) | 16% | 2.0% |
| Protease 1 (P6) | 10% | 1.7% |
| Protease 1 (P7) | 40% | 1.0% |

### Example 2: Example Formulations

The proteases according to the invention can be used and realize their effect in various washing and cleaning agent compositions.

TABLE 3
LIQUID WASHING AGENTS

| | Wt. % of active substance in the formulation | | | | | |
|---|---|---|---|---|---|---|
| Chemical name | A | B | C | D | E | F |
| Demineralized water | Remainder | Remainder | Remainder | Remainder | Remainder | Remainder |
| LAS | 5.5 | 20 | 15.0 | 5.5 | 21.7 | 23.5 |
| FAEOS | 7.0 | | 5.0 | | | |
| Palm kernel oleic acid | 3.0 | 8.0 | | | 7.0 | 7.4 |
| FAEO | 5.5 | | 8.0 | | | |
| C_{13/15} oxo alcohol, 8EO | | 25 | | | | |
| C₁₂₋₁₈ fatty alcohol | | | | | 22.4 | 23.4 |
| ethoxylate, 7EO | | | | | | |
| Alkyl polyglycoside | | | 4.0 | | | |
| Non-ionic surfactants | | | | 3.1 | | |
| Soap | | | 1.0 | 0.5 | | |
| HEDP | 0.5 | | | | | |
| DTPMPA 7Na | | 1.0 | 1.0 | 0.2 | 0.5 | 1.7 |
| Citric acid | 2.5 | | 3.0 | 0.23 | | |
| NaOH | 3.0 | | | 0.7 | | |
| Glycerol | 3.0 | 5.0 | | 0.5 | 9.4 | 10.2 |
| Ethanol | 1.5 | 3.0 | | | | 3.2 |
| 1,2-propanediol | | 10.0 | 12.0 | | 5.0 | 5.6 |
| Monoethanolamine | | 6.0 | 7.0 | | 6.0 | 6.1 |
| Boric acid | 1.0 | | 1.0 | 0.5 | | |
| Polyalkoxylated | | | | | 4.5 | |
| alkanolamine | | | | | | |
| Ethoxylated | | | | | 4.5 | 3.0 |
| polyethyleneimine | | | | | | |
| Protease | 6 HPE/ml | 6 HPE/ml | 6 HPE/ml | 6 HPE/ml | 6 HPE/ml | 6 HPE/ml |
| Fragrance(s) | 0.5 | 0.5 | 0.4 | 0.3 | 0.4 | 0.25 |
| DTI, SRP, other enzymes, | minors | minors | minors | minors | minors | minors |
| defoamers, etc. | | | | | | |

TABLE 4
SOLID WASHING AGENTS

| | Wt. % of active substance | | |
|---|---|---|---|
| | in the formulation | | |
| Chemical name | A | B | C |
| LAS | 12.2 | 12.0 | 10.1 |
| Sodium fatty alcohol sulphate, C₁₂₋₁₈ | 4.2 | | |
| Fatty alcohol, C₁₂₋₁₈, 7 EO | 4.1 | 2.3 | 1.5 |
| Soaps | 0.4 | | |
| Citrate | 2.0 | | |
| Sodium carbonate | 2.4 | 17.9 | 25.1 |
| Builders | 23.0 | 7.0 | 7.6 |
| Phosphonate | 1.2 | 1.1 | 1.2 |
| Polyacrylate | 0.12 | 2.8 | 3.0 |
| Carboxymethylcellulose | 2.3 | 2.0 | 1.1 |
| 2Na2 carbonate 3 H₂O₂ | 18.5 | 15.8 | |
| TAED | 10.9 | 3.5 | |
| Fragrance(s) | 0.5 | 0.3 | 0.4 |
| Protease | 6 HPE/ml | 6 HPE/ml | 6 HPE/ml |
| Sodium sulfate, foam inhibitor, | Remainder | Remainder | Remainder |
| optical brighteners, fragrances, other | | | |
| enzymes | | | |

TABLE 5
TWO-PHASE AUTOMATIC DISHWASHER DETERGENT

| Active substance content in wt. % | Powder phase | |
|---|---|---|
| (unless otherwise stated), based | (phase A) | |
| on the total weight of the powder phase | A1 | A2 |
| Sodium percarbonate | 13.0 | 15.0 |
| Non-ionic surfactants | 4.0 | 4.0 |
| Sulfonic acid group-containing polymer | 4.0 | 4.0 |
| HEDP (sodium salt) | 6.0 | 6.0 |
| Sodium carbonate (incl. sodium hydrogen | 24.0 | 28.0 |
| carbonate) | | |
| MGDA (trisodium salt) | 0 | 0 |
| Phyllosilicate (SKS 6 powder) | 4.0 | 4.0 |
| Sodium citrate (calculated as anhydrous sodium | 21.0 | 21.0 |
| citrate) | | |
| Amylase (Stainzyme ® Plus 24 Evity T; | 1.5 | 1.5 |
| indication as wt. % based on the amount of | | |
| preparation used, t.q.) | | |
| Protease (total active protein) | 40 mg/job | 40 mg/job |
| Misc (including perfume, dyes, preservatives, | Add 100 | Add 100 |
| fillers e.g., sodium sulfate, bleach catalyst | | |
| (MnTACN), bleach activator (TAED), zinc | | |
| acetate, silver protection, other enzymes) | | |
| Active substance content in wt. % | Gel phase | |
| (unless otherwise stated), based | (phase B) | |
| on the total weight of the gel phase | E1 | B2 |
| Polymer comprising acrylic acid-containing and | 10.0 | 11.0 |
| amidopropyl sulfonic acid-containing monomers | | |
| Glycerol | 27.0 | 25.0 |
| 1,3-propanediol | 30.0 | 30.0 |
| PEG 400 | 15.0 | 17.0 |
| PVOH | 15.0 | 14.0 |
| Misc (inter alia, process aids, pH adjusters, | Add 100 | Add 100 |
| perfume, dye) | | |
| Gelling time/min | less than 1 | less than 1 |

The phases A1 or A2 and the phases B1 or B2 can be combined with one another as desired. Total weight of both phases in a single portion of 18.5 g.

1. A protease that exhibits proteolytic activity, comprising:
an amino acid sequence that is at least 80% identical to the amino acid sequence indicated in SEQ ID NO:1 over its entire length, wherein the protease, in each case based on the numbering in accordance with SEQ ID NO:1, has at least one amino acid substitution selected from the group consisting of N121F, A194C, A209V, T218I, N237P and N237W.

2. The protease in accordance with claim 1, wherein the protease has an amino acid substitution combination selected from the group consisting of: (i) A209V-N237P, (ii) A209V, (iii) N121F-A209V-N237P, (iv) A194C-N218I-N237W, (v) A209V-N237W, and (vi) A209V-N218I-N237P, in each case based on the numbering in accordance with SEQ ID NO:1.

3. The protease of claim 1, further comprising:
(a) wherein the protease is obtained from a protease in accordance with claim 1 as starting molecule by single or multiple conservative amino acid substitution, wherein the protease, in each case based on the numbering in accordance with SEQ ID NO:1, has at least one amino acid substitution selected from the group consisting of N121F, A194C, A209V, T218I, N237P and N237W; or,
(b) wherein the protease is obtained from a protease in accordance with claim 1 as starting molecule by fragmentation or deletion, insertion or substitution mutagenesis and comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease, in each case based on the numbering in accordance with SEQ ID NO: 1, has at least one amino acid substitution selected from the group consisting of N121F, A194C, A209V, T218I, N237P and N237W.

4. A method for producing a protease, comprising:
introducing at least one amino acid substitution in at least one of the positions corresponding to positions 121, 194, 209, 218 and 237, based on the numbering in accordance with SEQ ID NO:1, selected from the group consisting of N121F, A194C, A209V, T218I, N237P and N237W, into a starting molecule that has an amino acid sequence that has at least 80% sequence identity with the amino acid sequence indicated in SEQ ID NO:1 over its entire length.

5. The method according to claim 4, further comprising one or more of the following method steps:
(a) introducing a single or multiple conservative amino acid substitution, wherein the protease, in each case based on the numbering in accordance with SEQ ID NO:1, has at least one amino acid substitution selected from the group consisting of N121F, A194C, A209V, T218I, N237P and N237W;
(b) altering the amino acid sequence by fragmentation or deletion, insertion or substitution mutagenesis such that the protease comprises an amino acid sequence that corresponds to the starting molecule over a length of at least 190, 200, 210, 220, 230, 240, 250, 255, 256, 257, 258, 259, 260, 261, 262, 263, 264, 265, 266, 267, 268 or 269 contiguous amino acids, wherein the protease, in each case based on the numbering in accordance with SEQ ID NO: 1, has at least one amino acid substitution selected from the group consisting of N121F, A194C, A209V, T218I, N237P and N237W.

6. A nucleic acid encoding a protease according to claim 1.

7. A vector containing a nucleic acid according to claim 6.

8. A non-human host cell that contains a protease according to claim 1 that secretes the protease into the medium surrounding the host cell.

9. A method for producing a protease, comprising
a) cultivating a host cell in accordance with claim 8 and
b) isolating the protease from the culture medium or from the host cell.

10. A washing or cleaning agent, that contains at least one protease according to claim 1.

11. A method for cleaning comprising,
using an agent according to claim 10 in at least one method step.


SEQUENCE LISTING (1 sequence)

SEQ ID NO: 1 - protein, 269 aa, Bacillus okhensis
NQTIPWGITRVQAPAAINRGFTGAGVRVAVLDTGISNHPDLNIRGGVSFVPGESTYQDGNGHGTHVAGTIAALNNSIGVVGVAPNTELYAVKVLGANGSGSISSIAQGLQWTAQNNIHVANLSLGSPTGSQTLELAVNQATSAGVLVVAASGNNGSGTISYPARYANALAVGATDQNNNRASFSQYGTGLNIVAPGVGVQSTYPGNRYASLSGTSMATPHVAGVAALVKQKNPGWSNTQIRQHLLNTATPLGSSNQYGSGLVNAEAATR